(19) United States
(12) Patent Application Publication
(10) Pub. No.: US 2014/0206763 A1
(43) Pub. Date: Jul. 24, 2014

(54) PHENYLBUTYRATE FOR TREATMENT OF SPORADIC INCLUSION-BODY MYOSITIS AND DISORDERS RELATING TO AUTOPHAGY IMPAIRMENT OR AMYLOID BETA 42 ACCUMULATION

(71) Applicant: UNIVERSITY OF SOUTHERN CALIFORNIA, Los Angeles, CA (US)

(72) Inventors: Valerie ASKANAS, Los Angeles, CA (US); Anna NOGALSKA, Glendale, CA (US); Carla D'AGOSTINO, Los Angeles, CA (US); William King ENGEL, Los Angeles, CA (US)

(73) Assignee: UNIVERSITY OF SOUTHERN CALIFORNIA, Los Angeles, CA (US)

(21) Appl. No.: 14/160,081

(22) Filed: Jan. 21, 2014

Related U.S. Application Data
(60) Provisional application No. 61/754,456, filed on Jan. 18, 2013.

(51) Int. Cl. A61K 31/216; A61K 31/192

(52) U.S. Cl. CPC A61K 31/216; A61K 31/192
USPC 514/533; 514/570; 435/375

(57) ABSTRACT

Disclosed is a method of improving lysosomal activity in a patient in need thereof including administering to the patient an effective amount of phenylbutyrate, preferably 4-phenylbutyrate in the form of at least one of sodium phenylbutyrate or glycerol phenylbutyrate. Also disclosed a method of improving lysosomal activity or ameliorating a pathological phenotype in a cell having impaired autophagy comprising treating the cell with phenylbutyrate, preferably 4-phenylbutyrate in the form of at least one of NaBP or GPB. The phenylbutyrate is generally administered in the form of a pharmaceutical composition including the phenylbutyrate and at least one carrier.

### CROSS-REFERENCE TO RELATED APPLICATIONS

This application claims the benefit of U.S. Provisional Application No. 61/745,456, filed Jan. 18, 2013, the entire contents of which are incorporated by reference in its entirety herein.

### BACKGROUND OF THE INVENTION

Inclusion body myositis (IBM) is a mainly degenerative muscle disease characterized by slowly progressive weakness and wasting of both distal and proximal muscles of the arms and legs. There are two general types of IBM: sporadic (s-IBM) and hereditary (h-IBM). A recent study has shown that current prevalence of s-IBM is at about 14.9 per million in the overall population with a prevalence of 51.3 per million among the 50-and-over age group.

The degenerative aspect of s-IBM is characterized by the appearance of vacuoles (“holes”) in the muscle fibers (cells), deposits of abnormal proteins within the muscle fibers and presence of filamentous inclusions (hence the name “inclusion body myositis”), containing phosphorylated tau. Its progressive course leads to severe disability, and handicap. As a result, patients are generally confined to wheelchairs, develop difficulties to feed themselves, and often have difficulty to swallow. Currently, there is no effective treatment for s-IBM.

As such, there is an urgent and continuing need for effective s-IBM treatments.

One aspect of the s-IBM muscle fiber is that its pathologic phenotype includes the abnormal accumulations intracellularly of Amyloid-beta42 (Aβ42) and its oligomers, and phosphorylated tau (Askanas et al., 1993, 2012; Askanas and Engel, 1998; Gouras et al., 2010; Honson and Kuret, 2008; Iqbal et al., 2010; Kannanayakal et al., 2008; Klein et al., 2004; LaFerla, 2010; LaFerla et al., 2007; Mirabella et al., 1996; Selkoe, 2011; Yankner and Lu, 2009). There is increasing evidence supported by experimental models employing cultured human muscle and transgenic mice (reviewed in Askanas and Engel, 2011; Askanas et al., 2012), that Aβ might be an important pathogenic aspect leading to s-IBM muscle-fiber degeneration, atrophy and weakness (reviewed by Askanas and Engel, 1998, 2011; Askanas et al., 2012; Dalakas, 2008).

Aβ42 is considered the most toxic specie of Aβ because of its high propensity to oligomerize, aggregate, and form amyloid fibrils (De Strooper, 2010; LaFerla et al., 2007). It is present within s-IBM muscle fibers in the form of both oligomers and aggregates (Nogalska et al., 2010a; Vattemi et al., 2009). Aβ is generated from the amyloid-β precursor protein cleaved by β- and γ-secretases (Chow et al., 2010; De Strooper, 2010; LaFerla et al., 2007), both of which are quantitatively increased in s-IBM muscle on the protein and mRNA levels (Nogalska et al., 2010c; Vattemi et al., 2001), as is the activity of γ-secretase (Nogalska et al., 2012), which is responsible for the Aβ42 generation (Chow et al., 2010; De Strooper, 2010).

Impaired autophagy is another important aspect of the s-IBM pathogenesis, as evidenced by the muscle-fiber vacuolization accompanied by: 1) inhibition of the lysosomal enzyme activities of cathepsins D and B (Nogalska et al., 2010b); 2) increase of LC3-II, a lipidated form of LC3 (Nogalska et al., 2010b), which is considered the most important indicator of increased autophagosome number, also occurring as the result of impaired autophagy (Klionsky et al., 2012); and 3) up-regulation of the proteasome-lysosome shuttle adaptor proteins NBR1 and p62 (D'Agostino et al., 2011; Nogalska et al., 2009). Both NBR1 and p62 bind to the ubiquitin moiety of proteins destined to be degraded (Lamark et al., 2009). Accumulation of selected autophagy-related proteins in s-IBM muscle fibers was also reported by others (Girolamo et al., 2013).

Phenylbutyrate (PB) is an orally bioavailable small molecule approved by the FDA for treatment of urea cycle-disorders (reviewed in Cuadrado-Tejedor et al., 2011; Iannitti and Palmieri, 2011; Papp and Csermely, 2006). In patients with urea-cycle disorders, phenylbutyrate (PB) (in the form of NaPB) is metabolized to phenylacetate, which conjugates with glutamine, and then as phenylacetylglutamine it scavenges ammonia to facilitate its excretion (Cuadrado-Tejedor et al., 2011; Iannitti and Palmieri, 2011; Monteleone et al., 2013). When taken daily and long-term by infants, children and adults, it is usually well tolerated (Iannitti and Palmieri, 2011). NaPB was also reported to be well-tolerated in preliminary trials of patients with amyotrophic lateral sclerosis (ALS) (Cudkowicz et al., 2009). Recently, glyceryl-phenylbutyrate (GPB), with action identical to sodium-phenylbutyrate. has been approved by the FDA for treating urea cycle-disorders. GPB was reported to be as effective as NaPB in lowering blood ammonia in those patients, and it avoids the undesirable load of sodium that accompanies treatment with NaPB (McGuire et al., 2010; Monteleone et al., 2013).

In addition to its action in urea-cycle disorders, PB is a histone-deacetylase (HDAC) inhibitor (reviewed in Cuadrado-Tejedor et al., 2011). PB also mimics the function of intracellular molecular chaperones by preventing protein aggregation, oligomerization and misfolding, and by reducing endoplasmic reticulum stress (Burrows et al., 2000; Kubota et al., 2006; Rubenstein et al., 1997).

### BRIEF SUMMARY OF THE INVENTION

One aspect of the present invention is our finding that phenylbutyrate (PB) treatment of experimentally autophagy-impaired cultured human muscle fibers substantially improved the phenotype of muscle-fibers by decreasing their vacuolization, substantially increased Cathepsin D and B activities, accompanied by a decrease of NBR1, p62 and LC3-II, substantially decreased Aβ42 and Aβ42 oligomers, and substantially decreased γ-secretase activity.

One embodiment of the present invention is a method for treating s-IBM, or ameliorating one or more pathological or clinical phenotypes of s-IBM comprising administering to a patient in need thereof an effective amount of phenylbutyrate. The phenylbutyrate can be administered in the form of sodium phenylbutyrate or glycerol-phenylbutyrate, or any other effective form of phenylbutyrate. Preferably, the phenylbutyrate would be as 4-phenylbutyrate.

Another embodiment of the present invention is method of improving lysosomal activity and/or ameliorating a pathological phenotype associated with impaired autophagy in a patient in need thereof, comprising administering to the patient an effective amount of phenylbutyrate. Preferably, the phenylbutyrate is 4-phenylbutyrate, and more preferably comprises at least one of sodium phenylbutyrate or glycerol-phenylbutyrate, or any other effective form of phenylbutyrate. Preferably, The pathological phenotype being present in muscle cells of the patient, the amount administered is to be effective for ameliorating the pathological phenotype in them. For example, the administered amount is effective to reduce, eliminate or slow the formation of vacuolization and/or other disease-induced detrimental changes in the muscle of the patient treated. Alternatively, the administered amount is effective (1) to reduce Aβ42 in the patient, or eliminate or slow the formation of Aβ42 in the patient; (2) to reduce Aβ42 oligomers in the patient, or slow or eliminate the formation of the formation of Aβ42 oligomers in the patient. Where the amount administered is effective to improve the lysosomal activity in muscle cells of the patient, the improved lysosomal activity is preferably an increase in an activity of at least one lysosomal protease selected from the group of Cathepsin D and Cathepsin B. and preferably both Cathepsin D and Cathepsin B.

These, and other aspects or embodiments of the present invention, are more fully described herein.

### BRIEF DESCRIPTION OF THE FIGURES

The file of this patent contains at least one drawing executed in color. Copies of this patent with color drawings will be provided by the Office upon request and payment of the necessary fee.

FIG. 1 shows that phenylbutyrate (PB) improves the pathologic phenotype and lysosomal functions of chloroquine-exposed cultured human muscle fibers (CHMFs). (A) Phase-contrast microscopy of living control “C”, chloroquine “Chl” exposed, and “Chl+PB”-treated CHMFs. While control “C” CHMFs appeared healthy and non-vacuolated, chloroquine exposure (Chl) induced pronounced vacuolization. However, NaPB treatment of Chl-exposed CHMFs virtually completely prevented their vacuolization (Chl+NaPB); in the upper three photos bar=50 μm; in the lower three photos bar=50 μm (the width of muscle fibers even in the same culture vary considerably). (B) GFP-LC3 fluorescence of control “C” CHMFs showed weak, diffuse LC3 cytoplasmic staining, whereas Chl-exposed cultures had multiple, bright green LC3-positive foci (‘puncta’). Treatment with NaPB largely prevented LC3 foci, the staining being mainly diffuse, with only rare GFP-LC3 positive foci detectable, bar=50 μm. (C) In three independent experiments the number of GFP-LC3 foci/puncta per muscle fiber was greatly increased in Chl-exposed CHMFs (8-fold, **p<0.01) whereas treatment with NaPB significantly prevented them (53%, *p<0.05). (D) Double labeling with GFP-LC3 (green fluorescence) and NBR1 (red fluorescence) showed that in Chl-exposed CHMFs there is a co-localization of LC3 and NBR1 foci and clusters, while controls are virtually negative, bar=65 μm. (E, F, G) Representative immunoblots and densitometric analyses based on nine separate experiments illustrate that in control “C” CHMFs, LC3-II (E), NBR1 (F), and p62 (G) are virtually undetectable or very low, but after Chl exposure they were significantly increased. Treatment of Chl-exposed CHMFs with NaPB caused the substantial decrease of all three components as compared to the Chl-alone exposed cultures (details and values in Results). For (E-G), representative immunoblots are below, and densitometric analyses are above. *** p<0.001. (H, I) Based on eight independent experiments, in CHMFs chloroquine inhibited activity of two major lysosomal proteases Cathepsin D and B; NaPB treatment of Chl-exposed CHMFs significantly increased activity of Cathepsin D (H) and Cathepsin B (I) (details in Results). In C and E-I, data are expressed as mean±SEM. *p<0.05, **p<0.01, ***p<0.001.

FIG. 2 shows that PB decreased Aβ42, Aβ42 oligomerization, and γ-secretase activity. (A) In five separate experiments, ELISA revealed that in chloroquine-exposed (Chl) CHMFs, Aβ42 was significantly increased; that increase was reversed by treatment with PB (Chl+NaPB) (details in Results). (B) Representative immunoblots showing Aβ42 oligomers. Protein loading was evaluated by the actin band. (B′) Densitometric analysis of Aβ42 oligomers based on seven separate experiments showed that NaPB significantly mitigated the increase of Aβ42 oligomers in “Chl+NaPB” treated cultures as compared to cultures exposed to “Chl” alone (details in Result). (C) In four independent experiments, as compared to control “C” CHMFs, Chloroquine (Chl)-exposed CHMFs had significantly increased γ-secretase activity, and that increased activity was significantly mitigated with NaPB treatment (Chl+NaPB) (details in Results). (Data are expressed as mean±SEM. *p<0.05, **p<0.01, ***p<0.001).

FIG. 3 shows the dose-dependent effect of PB on Control and Chloroquine-Exposed CHMFs. Exposure of Control CHMFs to increasing doses of NaPB, up to 15 mM, did not have any effect on either Cathepsin D (A) or Cathepsin B (B) activities, or on the protein levels of NBR1 (C), p62 (D) or LC3-II (E). However, in Chl-exposed CHMFs the 15 mM NaPB dose did significantly mitigate increase of NBR1 (C), and LC3-II (E); also both the 5 and 15 mM doses significantly decreased p62 (D). For (C-E), representative immunoblots are below, and densitometric analyses based on three separate experiments are above (details and all values in Results). Data are expressed as mean±SEM. *p<0.05, **p<0.01 (for the clarity of the presented data, the inter-group effects were not indicated)

FIG. 4 shows that PB improved lysosomal function and pathologic phenotype in bafilomycinAl-exposed CHMFs. (A, B) Based on eight independent experiments, bafilomycin (Baf) inhibited activity of two major lysosomal proteases, Cathepsin D and B in CHMFs; treatment of Baf-exposed CHMFs with NaPB increased activity of Cathepsin D (A) and Cathepsin B (B) (details and values in Results). (C) Phase-contrast microscopy of: living control “C”, “Baf-exposed (Baf)”, and “Baf+NaPB”-treated CHMFs. Whereas control “C” CHMFs appeared healthy and non-vacuolated, the bafilomycin exposure induced pronounced vacuolization similarly to that of chloroquine-exposed cultures. Treatment of Baf-exposed CHMFs with NaPB nearly completely prevented their vacuolization (Baf+NaPB), bar=40 μm. (D,E,F) Representative immunoblots and densitometric analyses, based on three separate experiments, show in control “C” CHMFs that LC3-II (D), NBR1 (E), and p62 (F), were virtually undetectable or very weak, whereas they were significantly increased after exposure to Baf—however, treatment of Baf-exposed CHMFs with NaPB caused their substantially-decreased elevation as compared to cultures exposed to “Baf” alone (details in Results). For (D-F) representative immunoblots are below, and densitometric analyses are above. Protein-loading was evaluated by the actin band.

(G) Representative immunoblots showing Aβ42 oligomers. Protein-loading was evaluated by the actin band. (G′) Densitometric analysis of Aβ42 oligomers, based on four separate experiments, showed that NaPB significantly mitigated increase of Aβ42 oligomers in “Baf+NaPB”-treated cultures. (H) ELISA, performed on two separate experiments, showed that Aβ42 was elevated in Baf-exposed CHMFs, and that increase was decreased by treatment with NaPB (Baf+NaPB) (details in Results). Data are expressed as mean±SEM, *p<0.05, **p<0.01, ***p<0.001.

### DETAILED DESCRIPTION OF THE INVENTION

### Abbreviations

Aβ42, amyloid beta 42;

Baf, bafilomycin Al;

CHMFs, cultured human muscle fibers;

Chl, chloroquine; HDAC, histone deacetylase;

iNOS, inducible nitric oxide synthase;

PB, phenylbutyrate

NaPB, sodium phenylbutyrate;

GPB, glycerol phenylbutyrate

s-IBM, sporadic inclusion-body myositis

### DEFINITIONS

Unless otherwise specifically defined herein, all technical and scientific terms used herein have the same meaning as commonly understood by one of ordinary skill in the art to which this invention pertains.

“Lysosome” refers to a membrane-enclosed compartment filled with soluble hydrolytic enzymes that control intracellular digestion of macromolecules. Lysosomes generally contain about 40 types of hydrolytic enzymes, including proteases, nucleases, glycosidases, lipases, phospholipases, phosphatases and sulfatases. Lysosomes perform many important cellular functions including phagocytosis and autolysis. Lysosomal enzymes are synthesized in the endoplasmic reticulum (ER), transported to the Golgi apparatus, where they are tagged for lysosomes by the addition of mannose-6-phosphate label. Malfunction of lysosomal enzymes can result in lysosomal storage diseases such as Tay-Sachs disease and acid-maltase deficiency (Pompe's disease).

“Autophagy” refers to a degradation pathway in lysosomes to dispose of obsolete parts of the cell itself. Without being limited to theory, the process appears to begin with the enclosure of an organelle by a double membrane, creating an authophagosome, which then fuses with a lysosome (or a late endosome). Once fused, digestion of the inner membrane of the autophagosome and its contents occurs.

A “vacuole” is a vesicle, often filled with fluid, in the cytoplasm of a cell.

“Cathepsin D” refers to Cathepsin D protein, EC 3.4.23.5, which is a protein that in humans is encoded by the CTSD gene. The CTSD gene encodes a lysosomal aspartyl protease composed of a protein dimer of disulfide-linked heavy and light chains, both produced from a single protein precursor.

“Cathepsin B” refers to Cathepsin B protein, EC 3.4.22.1, which is an enzymatic protein that in humans is encoded by the CTSB gene. The protein encoded by the CTSB gene is a lysosomal cysteine protease composed of a dimer of disulfide-linked heavy and light chains, both produced from a single protein precursor.

“γ-secretase” refers to a multi-subunit protease complex that cleaves single-pass transmembrane proteins at residues within the transmembrane domain.

“Aβ42” or “Aβ₁₋₄₂” herein refers to the amino acid sequence from amino acid position 1 to amino acid position 42 of the human amyloid β protein including both 1 and 42 and, in particular, refers to the amino acid sequence from amino acid position 1 to amino acid position 42 of the amino acid sequence DAEFRHDSGY EVHHQKLVFF AEDVGSNKGA IIGLMVGGW IA (corresponding to amino acid positions 1 to 42) or any of its naturally occurring variants. Such variants may be, for example, those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start of the Aβ peptide

The term “Aβ oligomer” herein refers to an association of Aβ peptides possessing distinct physical characteristics. The Aβ oligomers are stable, non-fibrillar, oligomeric assemblies.

As used herein, the term “patient” refers to any animal (e.g., a mammal), including, but not limited to, humans, non-human primates, rodents, and the like, which is to be the recipient of a particular treatment. Preferably, the “patient” is a human.

An “effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition are outweighed by the therapeutically beneficial effects.

The treatment methods of the present invention include the administration of phenylbutyrate, preferably 4-phenylbutyrate (IUPAC name: 4-phenylbutanoate), or a salt or ester thereof.

Butyrate, (also known as butanoate) is the traditional name for the conjugate base of butyric acid (also known as butanoic acid). The formula of the butyrate ion is C₄H₇O₂⁻. By way of example, sodium butyrate has the following formula (annotated with the carbon numbers in the IUPAC system):

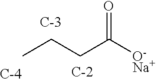

Phenylbutyrate, as that term is used herein, refers to butyrate (or a salt or ester thereof), in which at least one of the hydrogens at C-2, C-3 or C-4 is replaced with a phenyl group.

Preferably, the phenylbutyrate is 4-phenylbutyrate. More preferably, the 4-phenylbutyrate is in the form of at least one of Sodium Phenyl Butyrate or glycerol-phenylbutyrate. Sodium Phenyl Butyrate or “NaPB” refers to Sodium 4-phenylbutanoate, CAS No. 1716-12-7, having the formula:

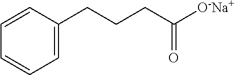

Glycerol-phenylbutyrate or “GPB” refers to 1,2,3-Propanetriyl tris(4-phenylbutanoate), CAS No. 611168-24-2, having the formula:

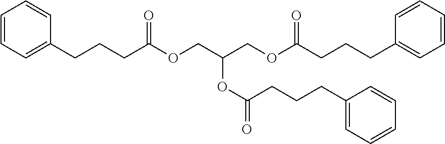 [SMILES: O=C(CCCc1ccccc1)OCC(COC(=O)CCCc1ccccc1)OC(=O)CCCc1ccccc1]

Glycerol-phenylbutyrate (GPB) contains the same active substance (4-phenylbutyrate) as NaPB but without the sodium and has been approved by the FDA to treat patients with urea-cycle disorders. GPB was reported to be as effective as NaPB in lowering blood ammonia in those patients, and it avoids the undesirable load of sodium that accompanies treatment with NaPB (McGuire et al., 2010; Monteleone et al., 2013).

The present invention includes the administration of phenylbutyrate, preferably 4-phenylbutyrate, and more preferably 4-phenylbutyrate either as NaBP or GPB to a patient in need thereof. It should be noted that NaPB is a salt, which may dissociate in aqueous media into its ionic components, which may themselves be hydrated or associated with other species in a specific biologic microenvironment. Further, it should also be noted that in patients with urea-cycle disorders, PB is metabolized to phenylacetate, which conjugates with glutamine, and then as phenylacetylglutamine it scavenges ammonia to facilitate its excretion. The present invention is intended to encompass and include the effect of phenylbutyrate and any of its metabolites. For instance, the present invention is intended to encompass NaPB or its ionic constituents in connection with the treatment methods of the present invention as well as any or all of its metabolites in the patient and/or cell to be treated.

One important aspect of this invention is a treatment for s-IBM patients, namely clinical improvement of the patients' muscle function or at least stopping or slowing progression of any already-present weakness. One aspect of the present invention is the finding that treatment with phenylbutyrate, such as NaPB or GPB, improves lysosomal activity and/or ameliorates pathological phenotypes in disorders associated with impaired autophagy. In connection with this finding, one aspect of the present invention is a method of improving lysosomal activity in a patient in need thereof and/or ameliorating a pathological phenotype associated with impaired autophagy in the patient, the method comprising administering to the patient an effective amount of phenylbutyrate, preferably 4-phenylbutyrate and more preferably at least one of NaPB or GPB, and also of any other effective form of phenylbutyrate. This method encompasses the action metabolites of phenylbutyrate generated after it is administered to the patient.

It is known that the lysosomal activity of muscle cells of patients with s-IBM is significantly decreased leading impaired autophagy (Nogalska et al 2010b). In a preferred embodiment, lysosomal activity is improved in the muscle cells of the patient by treatment with phenylbutyrate. In another embodiment, because the patient's muscle cells (i.e., the muscle fibers) are characterized by impaired autophagy, the pathological phenotype associated with impaired autophagy is present in those muscle cells, and the administered BP, including its metabolites, ameliorates one or more aspects of the pathological phenotype in the patient's muscle cells.

Another aspect of the present invention is a method of improving lysosomal activity or ameliorating a pathological phenotype in a cell having impaired autophagy comprising treating the cell with phenylbutyrate, preferably 4-phenylbutyrate, and more preferably at least one of NaPB or GPB. The location of the cell to be treated is not particularly limited, and this method includes native cells located in a human patient. The cell may be treated with phenylbutyrate for instance, by delivering it to the medium or the environment in which the cell is found, including but not limited to, the native environment of the cell within the human body. This method includes the action of metabolites of phenylbutyrate generated after it is introduced into the medium. The phenylbutyrate may be administered in the form of a pharmaceutical composition comprising the NaBP and/or GPB, or any other effective form of phenylbutyrate, and at least one carrier. Preferably, the cell is a muscle cell, and preferably a muscle-fiber cell.

While the exact molecular mechanisms underlying the protective effects of PB on the autophagic-lysosomal system are not known, its known properties as a molecular chaperone and as histone/protein decacetylase (HDAC) inhibitor (Cuadrado-Tejedor et al., 2011; Cuadrado-Tejedor et al., 2013; Iannitti and Palmieri, 2011) can be considered. Without being limited by theory, it is possible that the observed ameliorative effects of PB may relate mainly to its chaperone function, because treatment of CHMFs with trichostatinA, a widely used HDAC inhibitor, did not have an effect similar to that achieved with PB (data not shown).

In one embodiment of the invention, improving the lysosomal activity and/or ameliorating the pathological phenotype in the patient or cell to be treated is achieved by increasing the activity of at least one lysosomal protease in the patient or cell relative to untreated controls. Preferably, the lysosomal protease is one selected from the group of Cathepsin D and Cathepsin B. Preferably, the activity of both Cathepsin B and Cathespsin D are increased relative to controls. Without being limited to theory, PB has also been reported to ameliorate experimentally-induced endoplasmic reticulum-stress (ER-stress) in various cell lines (Cuadrado-Tejedor et al., 2011; Kubota et al., 2006), and ER-stress is known to be increased in muscle biopsies of s-IBM, v.i. Our results show that PB mitigates the reduction of Cathepsin B activity in one of our other models involving ER-stress-induced CHMFs (data not shown), in which we have previously demonstrated decreased activity of lysosomal proteases (Nogalska et al., 2010b). ER-stress has been demonstrated in s-IBM patients' muscle fibers (Nogalska et al., 2006; Vattemi et al., 2004).

In another embodiment of the invention, ameliorating the pathological phenotype in the patient or cell to be treated is to be evidenced by reducing the number of vacuoles, or eliminating or slowing the formation of vacuoles (relative to untreated controls) in the cells of the patient treated or in the cells to be treated.

In another embodiment of the invention, ameliorating the pathological phenotype in the patient or cell to be treated is accomplished by reducing Aβ42, or eliminating or slowing the formation of Aβ42 (relative to untreated controls) in the cells of the patient treated or in the cells to be treated.

In another embodiment of the invention, ameliorating the pathological phenotype in the patient or cell to be treated is accomplished by reducing Aβ42 oligomers, or slowing or eliminating the formation of the formation of Aβ42 oligomers (relative to untreated controls) in the cells of the patient treated or in the cells to be treated.

In one embodiment of the invention, improving the lysosomal activity and/or ameliorating the pathological phenotype in the patient or cell to be treated is achieved by, for instance, decreasing the activity of γ-secretase, which produces Aβ42, in muscle cells of the PB-treated patient.

Without being limited to theory, the effect of PB minimizing the Aβ42 increase might be occurring through: a) improving Aβ42 degradation by our demonstrated increased activities of Cathepsin D and B, which are known to degrade Aβ42 (Saido and Leissring, 2012); b) lowering production of Aβ42 through the demonstrated decreased activity of γ-secretase, an enzyme responsible for Aβ42 generation (Chow et al., 2010; De Strooper, 2010), and whose activity in our system appears to depend on lysosomal activity (Nogalska et al., 2012); or both.

The distinguishing pathological characteristics of s-IBM are the accumulation of multi-aggregates in vacuolated muscle fibers, including Aβ42 and its known toxic oligomers, as well as phosphorylated tau. Another aspect of the present invention is based on the inventors' experimental model of s-IBM comprising cultured human muscle fibers (CHMFs) in which autophagy is experimentally inhibited with a concurrent increase of Aβ42 and its oligomers. In this experimental model, treatment with PB decreased Aβ42 and its oligomers.

Another aspect of the present invention is a method of treating s-IBM or ameliorating one or more pathological phenotypes of s-IBM comprising administering to a patient in need thereof an effective amount of a pharmaceutical composition comprising phenylbutyrate, preferably 4-phenylbutyrate, and more preferably one or more of sodium phenyl butyrate or glycerol-phenylbutyrate. Preferably, the phenylbutyrate is administered in an amount effective to improve at least one measure of muscle performance, such as muscle strength or muscle endurance. The nature of the test is not particularly limited, and a number of such tests are well known to those of ordinary skill in the art. In an additional embodiment, the phenylbutyrate is administered in an amount effective to accomplish at least one of the following: (1) Reducing, eliminating or slowing the formation of vacuolization (relative to untreated controls) in the muscle cells of the patient treated; (2) reducing Aβ42, or eliminating or slowing the formation of Aβ42 (relative to untreated controls) in the cells of the patient (3) reducing Aβ42 oligomers, or slowing or eliminating the formation of the formation of Aβ42 oligomers (relative to untreated controls) in the cells of the patient treated or in the cells to be treated; (4) decreasing the activity of γ-secretase in a cell of the patient treated; (5) increasing the activity of Cathepsin D in the cells of the patient to be treated; and/or (6) increasing the activity of Cathepsin B in the cell of the patient treated.

### Chronic Administration

In practicing the methods described herein, phenylbutyrate may be periodically re-administered during the time period for which treatment is desired. Periodic re-administration generally includes the administration of more than one dose of an agent over a period of time, and may include regular administration for an extended period of time. Periodic re-administration may include chronic administration, which is the administration of therapy over a prolonged period of time, in some cases, for the duration of a subject's lifetime following the initial administration. During periodic re-administration, including chronic administration, the concentration of the therapeutic agent is maintained at a therapeutically or prophylactically effective level throughout the course of treatment.

### Pharmaceutical Compositions

Phenylbutyrate can be formulated as pharmaceutical compositions and administered to a patient, such as a human patient, in a variety of forms adapted to the chosen route of administration, i.e., orally or parenterally, by intravenous, intramuscular, topical or subcutaneous routes.

The phenylbutyrate composition may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. The percentage of the compositions and preparations may, of course, be varied. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

### EXPERIMENTAL, OVERVIEW

The materials, methods, and examples disclosed herein are illustrative only and not intended to be limiting. Other features, objects, and advantages of the invention will be apparent from the description and the accompanying drawings, and from the appended claims.

Strong Similarities Between the S-IBM Patient's Muscle-Fiber Abnormalities and the Changes in the Muscle-Fibers of Our Induced Experimental Model.

An important aspect related to this invention is that cultured human muscle fibers with experimentally impaired autophagy exactly reproduce the following phenotypic pathologic and biochemical abnormalities present in s-IBM patients' muscle biopsies, as follows:
a) Pronounced muscle-fiber deterioration, as evidenced by multiple vacuoles, and lysosomal abnormalities manifest both by light microscopy and electron-microscopy;
b) Decreased activity of lysosomal proteases, Cathepsin D and Cathepsin B;
c) Increased autophagosomes as evidenced by increased LC3 both by immunostaining and immunoblots;
d) Increased proteasomal-lysosomal shuttle proteins, NBR1 and p62
e) Increased Aβ42 by Elisa, and Aβ42-oligomers by immunoblots;
f) Increased gamma-secretase, an enzyme involved in Aβ42 production.

Accordingly, this experimental in vitro model of s-IBM has proven to be an excellent model for testing drugs, which might be used for treating patients with s-IBM.

### Detailed Discussion of Certain Results

PB Reduced the Consequences of Impaired Autophagy in CHMFs.

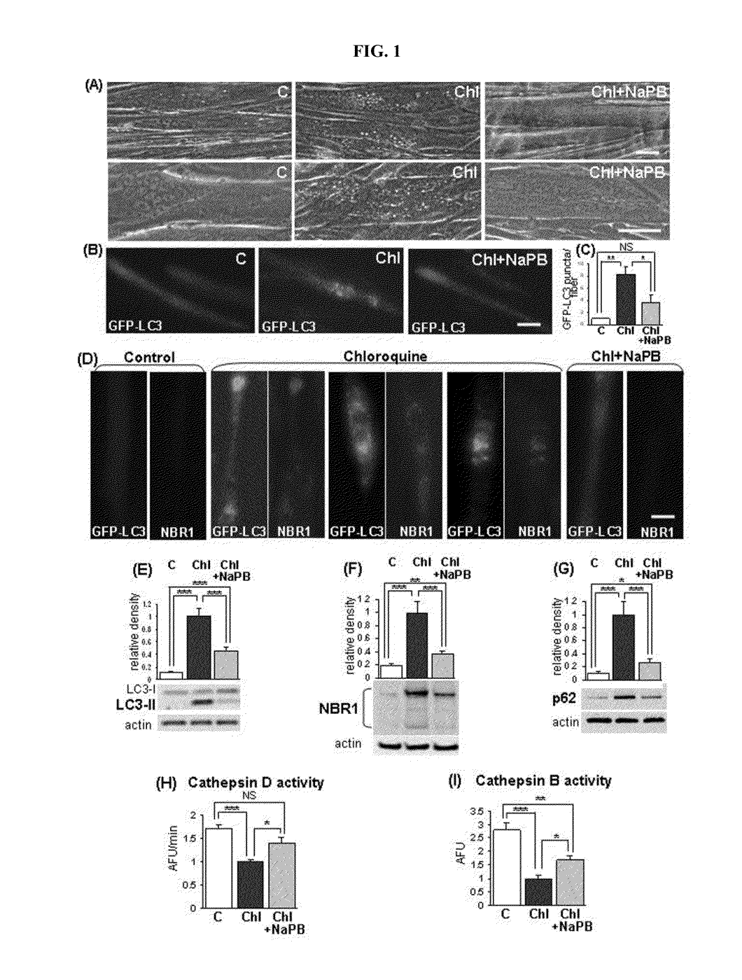

Treatment with PB diminished development of the abnormal phenotype of our experimental model of s-IBM, viz. chloroquine-exposed CHMFs, by prominently eliminating their vacuolization (FIG. 1A). This was associated with a decrease of morphologically detectable LC3 (FIG. 1B). For example, in GFP-LC3 transduced control-CHMFs, LC3 was detected as a very weak and diffuse cytoplasmic staining, whereas chloroquine-exposed cultures contained multiple bright green-fluorescent LC3-positive foci, often present in large clusters (FIG. 1B). (Transduction of chloroquine-exposed CHMFs with a mutated LC3-GFP resulted in only GFP-background staining [not shown]). By contrast, in chloroquine+NaPB-treated cultures, the majority of fibers, similarly to the appearance in controls, had only a diffuse cytoplasmic staining and only small GFP-LC3 foci present in occasional fibers (FIG. 1B), whose number was significantly decreased 53% (p<0.05) as compared to those in chloroquine-only exposed cultures (FIG. 1C). Morphologic data regarding LC3 paralleled those obtained by immunoblots, showing 54% (p<0.001) decrease of LC3-II in chloroquine+NaPB treated cultures, as compared to CHMFs exposed to chloroquine alone (FIG. 1E) (the data were normalized to chloroquine-only exposed cultures). In our current experiments, exposure of CHMFs to chloroquine significantly increased LC3-II (9-fold, p<0.001)(FIG. 1E), in line with previously published results (Nogalska et al., 2010b). (Unexposed-control CHMFs had only marginal amounts of LC3-II [FIG. 1E]). Accordingly, association of the GFP-LC3 fluorescence pattern with the decrease of LC3-II on immunoblots suggests that NaPB improved, viz. increased, lysosomal degradation in autophagy-impaired CHMFs.

Similarly to what has been demonstrated previously (D'Agostino et al., 2011), in our current experiments, exposure to chloroquine significantly increased NBR1 levels (5-fold, p<0.001), but NaPB treatment of those autophagy-impaired CHMFs reduced their increased NBR1 by 63% (p<0.001) (FIG. 1F), again suggesting improved autophagic degradation. This corresponds to our immunohistochemical studies, which demonstrated that autophagy-impaired CHMFs had numerous various-sized NBR1-immuno-positive aggregates that co-localized with GFP-LC3 clusters, while NaPB treatment of the chloroquine-inhibited CHMFs had only a very weak and diffuse NBR1-immunoreactivity (FIG. 1D). Uninhibited-control CHMFs had only minimal amounts of NBR1 by immunoblots, and virtually negative immunofluorescence staining (FIG. 1D,F). Like NBR1, in our current experiments p62 was substantially increased in CHMFs with impaired autophagy (9-fold, p<0.001) (FIG. 1G), in line with what was reported previously (Nogalska et al., 2009). And in autophagy-impaired CHMFs, treatment with NaPB caused 74% (p<0.001) decrease of p62 (FIG. 1G).

PB Improved Cathepsin D and B Activities.

Autophagy-impaired CHMFs treated with PB had significantly increased activities of both Cathepsin D and B (FIG. 1H,I), which corresponded to their decreased vacuolization and decreased LC3-II, NBR1 and p62. As reported previously (Nogalska et al., 2010b), and confirmed in our current experiments, chloroquine-exposed CHMFs had significantly reduced activities of Cathepsin D and B (Cathepsin D 40%, p<0.001, and Cathepsin B 60% p<0.001) (FIG. 1H,I), however, treatment with NaPB increased Cathepsin D 40% (p<0.05), and Cathepsin B 70% (p<0.05) activities (FIG. 1H, I).

PB Decreased Aβ42, its Oligomerization, and γ-Secretase Activity.

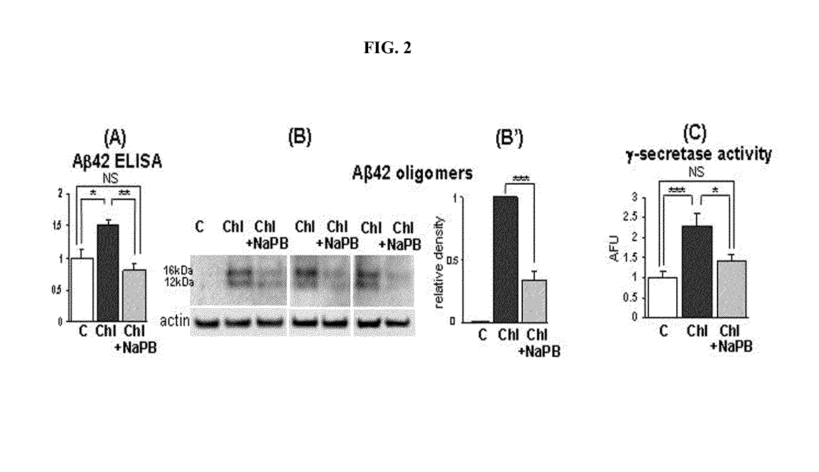

In CHMFs with chloroquine-impaired-autophagy, Aβ42 was increased 60% (p<0.05) by ELISA as compared to control CHMFs (FIG. 2A). PB (as NaPB) treatment of the chloroquine-inhibited cultures decreased Aβ42 to levels similar to controls (53%, p<0.01) (FIG. 2A). Aβ42 oligomers, which are virtually not detectable in control cultures but are prominent in autophagy-impaired CHMFs (FIG. 2B), were per immunoblots decreased 65% (p<0.001) by PB treatment as compared to autophagy-impaired but NaPB-non-treated CHMFs (FIG. 2B-B′). In autophagy-impaired cultures, NaPB treatment also decreased γ-secretase activity by 56% (p<0.05) as compared to untreated autophagy-impaired CHMFs (FIG. 2C).

Dose-Dependent Effects of PB in Control and Chloroquine-Exposed CHMFs.

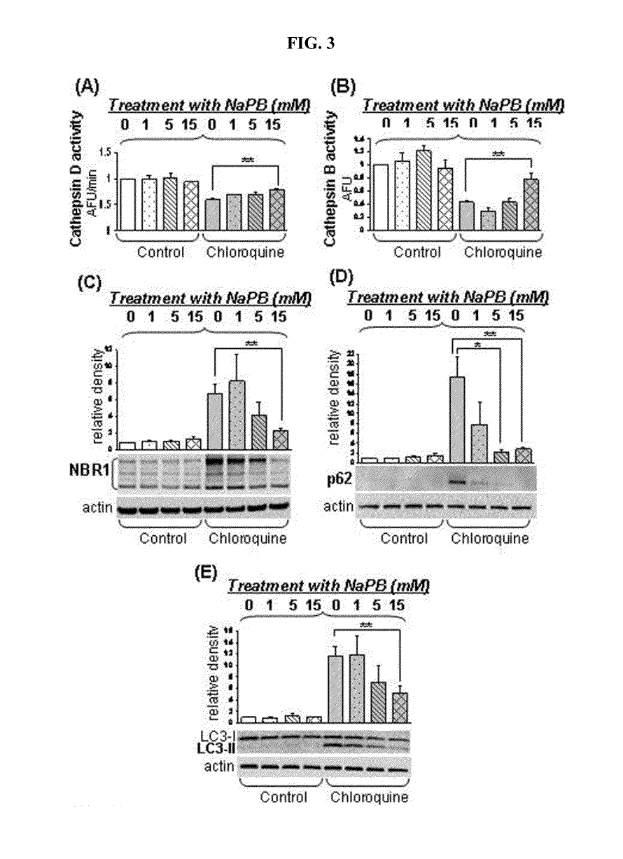

Treatment of control CHMFs with different doses of NaPB (1-15 mM) did not exert any effect on the parameters evaluated in this study. For example, neither the activities of Cathepsin D and B were changed (FIG. 3A,B); nor were the protein levels of NBR1, p62 or LC3-II altered (FIG. 3C,D, E, respectively). Even though an effect of lower doses of NaPB (less than 15 mM) in autophagy-impaired CHMFs was noticeable in some parameters studied, only p62 was statistically significantly decreased, at a dose 5 mM (FIG. 3D). Whether lower doses of NaPB applied to CHMFs for a longer period of time than in this study would have a beneficial effect on some of the parameters studied here is not known. (The limited amount of human muscle material from which our primary cultures are derived did not permit long-term experiments involving lower doses of NaPB.)

PB Also Increased Activity of Lysosomal Cathepsins and Ameliorated the Consequences of Impaired Autophagy in BafilomycinAl-Exposed CHMFs.

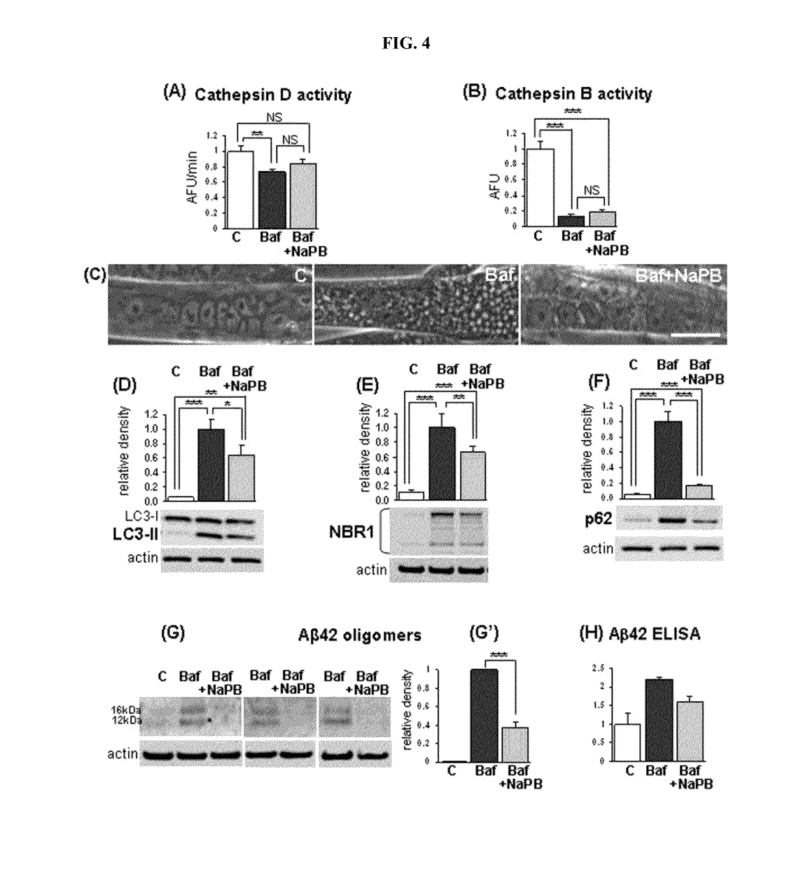

To evaluate whether the beneficial effect of PB on CHMFs with chloroquine-impaired autophagy might relate narrowly to an PB interference with chloroquine action, we also used another CHMFs model in which autophagy was impaired by exposure to bafilomycinAl (an inhibitor of lysosomal activity that impairs lysosomal degradation through a mechanism different than that produced by chloroquine [see above]) (Klionsky et al., 2012; Shacka et al., 2006). Similarly to our previous studies (Nogalska et al., 2010b), in our current experiments bafilomycin significantly reduced activities of Cathepsin D and B (25%, p<0.01, and 85%, p<0.001, respectively) (FIG. 4A,B) as compared to untreated controls. Concurrent treatment of these bafilomycin-inhibited cultures with NaPB resulted in definitely higher activities of Cathepsin D (15%), and Cathepsin B (40%) (FIG. 4 A,B) compared to ones without NaPB, but they did not quite reach the control levels. These results co-occurred with a morphologically prominent decrease of their vacuolization (FIG. 4C). These results in bafilomycin-treated CHMFs, also correspond to the NaPB effects of decreased LC3-II (36%, p<0.05; FIG. 4D), NBR1 (34%, p<0.01; FIG. 4E), and p62 (83%, p<0.001; FIG. 4F)—all of which were otherwise greatly increased in non-NaPB treated cultures due to bafilomycin-exposure (17-fold, p<0.01, 9-fold, p<0.001, and 18-fold, p<0.001 respectively) (FIG. 4D-F).

In addition, similarly to NaPB-influence on chloroquine-exposed cultures, NaPB-treatment of bafilomycin-exposed cultures led to substantially lower amounts of Aβ42 oligomers (60% less, p<0.001; FIG. 4G,G′), and of Aβ42 (28% less, FIG. 4H; statistical significance was not reached because the ELISA experiment was performed on only 2 culture sets).

In both types of autophagy-impaired cultures, the ANOVA test indicated that NaPB treatment clearly improved all the studied variables. The best results were in Cathepsin D, Aβ42 by ELISA, and γ-secretase activity, whose values after NaPB treatment were “fully corrected” (i.e., not significantly worse than controls). The other values were also prominently improved, but not fully reversed (i.e., still significantly different from untreated controls).

### Experimental Methods, Details

Cultured Human Muscle Fibers (CHMFs).

Primary cultures of normal human muscle were established as routinely performed in our laboratory (Askanas and Engel, 1992) from archived satellite cells of portions of diagnostic muscle biopsies from patients who, after all tests were performed, were considered free of muscle disease. Each experiment was performed on 3-9 different culture sets, each established from satellite cells derived from a different muscle biopsy. 10-15 days after myoblast fusion, the well-differentiated myotubes were, as previously described (Nogalska et al., 2012; Nogalska et al., 2010b), exposed for 24 hours to either: a) chloroquine (Chl), a lysosomotropic agent raising lysosomal pH and inhibiting activities of cathepsins (Shacka et al., 2006) (50 μM, Sigma-Aldrich, St. Louis, Mo.); or b) bafilomycin Al (Baf), an inhibitor of lysosomal V-ATPase causing an increase in lysosomal pH and inhibiting activities of cathepsins (Shacka et al., 2006) (25 nM, Sigma-Aldrich). CHMFs were also treated with NaPB (Tocris/R&D Systems, Minneapolis, Minn.) in doses ranging from 1-15 mM, used either alone or together with chloroquine or bafilomycin. After several preliminary experiments, for final-treatment experiments we selected a 15 mM dose of NaPB, because in control CHMFs even this relatively high dose did not produce either adverse morphologic abnormalities or affect muscle-fiber viability, and when applied together with chloroquine or bafilomycin exerted a consistently significant beneficial effect.

To morphologically evaluate accumulation of autophagosomes, three different tissue-culture sets (experiments) were transduced with PremoAutophagy Sensor GFP-LC3 (Invitrogen, Carlsbad, Calif.) according to the manufacturer's instructions. 24 hours later, CHMFs were treated with chloroquine as described above. Counting of the GFP-LC3 puncta in the muscle fibers was performed on all three different culture experiments. Each experiment involved the same three different conditions: control, chloroquine-exposed, and chloroquine-exposed-plus-NaPB-treated cultures. In each experiment, each condition was composed of two cultures. For the analysis, eight random fields per condition of each culture were photographed (magnification ×40) in each of the three experiments. Overall, in each of the three conditions GFP-LC3 puncta were evaluated in more than 80 fibers, independently by two investigators (AN and CD). In addition, GFP-LC3 transduced CHMFs were also processed for immunofluorescence of NBR1 (1:100, 4BR, Santa Cruz Biotechnology, Santa Cruz, Calif.).

Immunoblots.

CHMFs were harvested in RIPA buffer and immunoblotted, as previously detailed (D'Agostino et al., 2011; Nogalska et al., 2012; Nogalska et al., 2010b) using antibodies against LC3 (1:500, Novus Biologicals, Littleton, Colo.), p62 (1:100, A-6, Santa Cruz Biotechnology) or NBR1 (1:700). Aβ42 oligomers were studied using 6E10 antibody (1:500, Covance, Princeton, N.J.), as described (Nogalska et al., 2010a). Blots were developed using anti-rabbit or anti-mouse WesternBreeze chemiluminescence kits (Invitrogen). Protein loading was evaluated by the actin band (1:600, C-2, Santa Cruz Biotechnology). Quantification of the immunoreactivity was performed by densitometric analysis using NIH Image J 1.310 software.

Measurement of Cathepsin D and B Activities.

Cathepsin D [EC 3.4.23.5] activity was measured utilizing a Cathepsin D Assay Kit (Sigma, St. Louis, Mo.) according to manufacturer's instructions and as previously detailed (Nogalska et al., 2010b). Homogenates were prepared in phosphate buffer-saline with addition of CHAPS (0.05%) (Sigma-Aldrich). All samples were measured in duplicate. The fluorescence emission excited at 355 nm was recorded at 405 nm every 2 minutes. Activity of cathepsin D measured in the linear phase of the reaction was expressed as fluorescence-intensity per minute.

Cathepsin B [EC 3.4.22.1] activity was measured as described (Nogalska et al., 2010b). Samples were measured in duplicate. All samples were incubated at 37° C. for 45 min, and the fluorescence emission excited at 355 nm was recorded at 444 nm. Activity of cathepsin B was expressed as arbitrary fluorescence units.

Aβ42 Enzyme-Linked Immunosorbent Assay (ELBA).

To quantify endogenous Aβ42 content in cultured human muscle fibers, we used the “Human β-Amyloid (1-42) ELISA kit, WAKO, High Sensitive” (WAKO Chemicals USA, Inc., Richmond, Va.) as described (Nogalska et al., 2012). Homogenates were freshly prepared in RIPA buffer containing a protease inhibitor cocktail (Roche Diagnostic, Mannheim, Germany). 100 μg of protein per well from each sample were diluted in a Standard Diluent Buffer provided in the kit, added to the assay plates in duplicate, and incubated overnight at 4° C. The ELISA was performed the next day, according to manufacturer's protocol. The absorbance was read at 450 nm. (We previously demonstrated that Aβ40 is not detectable in CHMFs [Nogalska et al., 2012].)

Measurement of γ-Secretase Activity.

γ-secretase activity was measured as previously described (Nogalska et al., 2012). Briefly, cultures were homogenized in PBS buffer with addition of 0.05% CHAPS. The supernatants containing 50 μg of protein were added to the reaction mixture containing assay buffer (50 mM TRIS-HCl pH 6.8, 2 mM EDTA, 0.25% CHAPS). The reaction was initiated by adding a specific synthetic substrate NMA-Amyloid β-Protein (aa708-715)-Lys-(DNP) Arg-Arg-Arg-amide trifluoroacetate salt (10 μM) (Sigma-Aldrich). After overnight incubation at 37° C., the samples were centrifuged at 16,000×g for 15 minutes, supernatants were transferred to a 96-well plate and the fluorescence emission excited at 390 nm was recorded at 444 nm. Activity of γ-secretase was expressed as arbitrary fluorescence units.

Statistical Analysis.

For all experiments, except Aβ42 oligomers, the statistical significance was determined by one-way analysis of variance (ANOVA) followed by Tukey-Kramer post-hoc multiple comparison test, using GraphPad InStat software. The statistical significance between two groups (Aβ42 oligomers) was determined by Student's t-test. The level of significance was set at p<0.05. Data are presented as means±SEM.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Those skilled in the art will recognize, or be able to ascertain using no more than routine experimentation, many equivalents to the specific embodiments of the method and compositions described herein. Such equivalents are intended to be encompassed by the following claims.

### REFERENCES

All of the references herein, including the foregoing, are hereby incorporated herein by reference in their entirety.
Askanas, V., Alvarez R. B., Engel W. K., 1993. Beta-amyloid precursor epitopes in muscle fibers of inclusion body myositis. Ann Neurol. 34, 551-60.
Askanas, V., Engel, W. K., Cultured normal and genetically abnormal human muscle. In: L. P. Rowland, S. Di Mauro, (Eds.), The handbook of clinical neurology, myopathies. North Holland, Amsterdam, 1992, pp. 85-116.
Askanas, V., Engel, W. K., 1998. Does overexpression of betaAPP in aging muscle have a pathogenic role and a relevance to Alzheimer's disease? Clues from inclusion body myositis, cultured human muscle, and transgenic mice. Am J. Pathol. 153, 1673-7.
Askanas, V., Engel, W. K., 2011. Sporadic inclusion-body myositis: Conformational multifactorial ageing-related degenerative muscle disease associated with proteasomal and lysosomal inhibition, endoplasmic reticulum stress, and accumulation of amyloid-beta42 oligomers and phosphorylated tau. Presse Med. 40, e219-35.
Askanas, V., Engel W. K., Nogalska A., 2012. Pathogenic considerations in sporadic inclusion-body myositis, a degenerative muscle disease associated with aging and abnormalities of myoproteostasis. J Neuropathol Exp Neurol. 71, 680-93.
Broccolini, A., Engel W. K., Alvarez R. B., Askanas V., 2000. Redox factor-1 in muscle biopsies of patients with inclusion-body myositis. Neurosci Lett. 287, 1-4.
Brunetti-Pierri, N., Lanpher B., Erez A., Ananieva E. A., Islam M., Marini J. C., et al., 2011. Phenylbutyrate therapy for maple syrup urine disease. Hum Mol Genet. 20, 631-40.
Burrows, J. A., Willis L. K., Perlmutter D. H., 2000. Chemical chaperones mediate increased secretion of mutant alpha 1-antitrypsin (alpha 1-AT) Z: A potential pharmacological strategy for prevention of liver injury and emphysema in alpha 1-AT deficiency. Proc Natl Acad Sci USA. 97, 1796-801.
Chow, V. W., Mattson M. P., Wong P. C., Gleichmann M., 2010. An overview of APP processing enzymes and products. Neuromolecular Med. 12, 1-12.
Cuadrado-Tejedor, M., Garcia-Osta A., Ricobaraza A., Oyarzabal J., Franco R., 2011. Defining the mechanism of action of 4-phenylbutyrate to develop a small-molecule-based therapy for Alzheimer's disease. Curr Med Chem. 18, 5545-53.
Cuadrado-Tejedor, M., Ricobaraza A. L., Torrijo R., Franco R., Garcia-Osta A., 2013. Phenylbutyrate is a multifaceted drug that exerts neuroprotective effects and reverses the Alzheimer s disease-like phenotype of a commonly used mouse model. Curr Pharm Des 19, 5076-5084.
Cudkowicz, M. E., Andres P. L., Macdonald S. A., Bedlack R. S., Choudry R., Brown R. H., Jr., et al., 2009. Phase 2 study of sodium phenylbutyrate in ALS. Amyotroph Lateral Scler. 10, 99-106.
D'Agostino, C., Nogalska A., Cacciottolo M., Engel W. K., Askanas V., 2011. Abnormalities of NBR1, a novel autophagy-associated protein, in muscle fibers of sporadic inclusion-body myositis. Acta Neuropathol. 122, 627-36.
Dalakas, M. C., 2008. Interplay between inflammation and degeneration: using inclusion body myositis to study “neuroinflammation”. Ann Neurol. 64, 1-3.
Dalakas, M. C., 2011. Review: An update on inflammatory and autoimmune myopathies. Neuropathol Appl Neurobiol. 37, 226-42.
De Strooper, B., 2010. Proteases and proteolysis in Alzheimer disease: a multifactorial view on the disease process. Physiol Rev. 90, 465-94.
Dimachkie, M. M., Barohn, R. J., 2013. Inclusion body myositis. Curr Neurol Neurosci Rep. 13, 321.
Engel, W. K., Askanas, V., 2006. Inclusion-body myositis: clinical, diagnostic, and pathologic aspects. Neurology. 66, S20-9.
Girolamo, F., Lia A., Amati A., Strippoli M., Coppola C., Virgintino D., et al., 2013. Overexpression of autophagic proteins in the skeletal muscle of sporadic inclusion body myositis. Neuropathol Appl Neurobiol. doi:10.1111/nan.12040.
Gouras, G. K., Tampellini D., Takahashi R. H., Capetillo-Zarate E., 2010. Intraneuronal beta-amyloid accumulation and synapse pathology in Alzheimer's disease. Acta Neuropathologica. 119, 523-41.
Honson, N. S., Kuret, J., 2008. Tau aggregation and toxicity in tauopathic neurodegenerative diseases. J Alzheimers Dis. 14, 417-22.
Iannitti, T., Palmieri, B., 2011. Clinical and experimental applications of sodium phenylbutyrate. Drugs in R&D. 11, 227-49.
Iqbal, K., Wang, X., Blanchard, J., Liu, F., Gong, C. X., Grundke-Iqbal, I., 2010. Alzheimer's disease neurofibrillary degeneration: pivotal and multifactorial. Biochem Soc Trans. 38, 962-6.
Kannanayakal, T. J., Mendell, J. R., Kuret, J., 2008. Casein kinase 1 alpha associates with the tau-bearing lesions of inclusion body myositis. Neurosci Lett. 431, 141-5.
Klein, W. L., Stine W. B., Jr., Teplow D. B., 2004. Small assemblies of unmodified amyloid beta-protein are the proximate neurotoxin in Alzheimer's disease. Neurobiol Aging. 25, 569-80.
Klionsky, D. J., Abdalla F. C., Abeliovich H., Abraham R. T., Acevedo-Arozena A., Adeli K., et al., 2012. Guidelines for the use and interpretation of assays for monitoring autophagy. Autophagy. 8, 445-544.
Kubota, K., Niinuma Y., Kaneko M., Okuma Y., Sugai M., Omura T., et al., 2006. Suppressive effects of 4-phenylbutyrate on the aggregation of Pael receptors and endoplasmic reticulum stress. J. Neurochem. 97, 1259-68.
LaFerla, F. M., 2010. Pathways linking Abeta and tau pathologies. Biochem Soc Trans. 38, 993-5.
LaFerla, F. M., Green K. N., Oddo S., 2007. Intracellular amyloid-beta in Alzheimer's disease. Nat Rev Neurosci. 8, 499-509.
Lamark, T., Kirkin V., Dikic I., Johansen T., 2009. NBR1 and p62 as cargo receptors for selective autophagy of ubiquitinated targets. Cell Cycle. 8, 1986-90.
McGuire, B. M., Zupanets I. A., Lowe M. E., Xiao X., Syplyviy V. A., Monteleone J., et al., 2010. Pharmacology and safety of glycerol phenylbutyrate in healthy adults and adults with cirrhosis. Hepatology. 51, 2077-85.
Mirabella, M., Alvarez R. B., Bilak M., Engel W. K., Askanas V., 1996. Difference in expression of phosphorylated tau epitopes between sporadic inclusion-body myositis and hereditary inclusion-body myopathies. J Neuropathol Exp Neurol. 55, 774-86.
Monteleone, J. P., Mokhtarani M., Diaz G. A., Rhead W., Lichter-Konecki U., Berry S. A., et al., 2013. Population pharmacokinetic modeling and dosing simulations of nitrogen-scavenging compounds: disposition of glycerol phenylbutyrate and sodium phenylbutyrate in adult and pediatric patients with urea cycle disorders. J Clin Pharmacol. 53, 699-710.
Nogalska, A., D'Agostino C., Engel W. K., Askanas V., 2011. Novel demonstration of conformationally-modified tau in sporadic inclusion-body myositis muscle fibers. Neurosci Lett. 503, 229-233.
Nogalska, A., D'Agostino C., Engel W. K., Askanas V., 2012. Activation of the gamma-secretase complex and presence of gamma-secretase-activating protein may contribute to Abeta42 production in sporadic inclusion-body myositis muscle fibers. Neurobiol Dis. 48, 141-9.
Nogalska, A., D'Agostino C., Engel W. K., Klein W. L., Askanas V., 2010a. Novel demonstration of amyloid-beta oligomers in sporadic inclusion-body myositis muscle fibers. Acta Neuropathol. 120, 661-6.
Nogalska, A., D'Agostino C., Terracciano C., Engel W. K., Askanas V., 2010b. Impaired autophagy in sporadic inclusion-body myositis and in endoplasmic reticulum stress-provoked cultured human muscle fibers. Am J. Pathol. 177, 1377-1387.
Nogalska, A., Engel W. K., Askanas V., 2010c. Increased BACE1 mRNA and noncoding BACE1-antisense transcript in sporadic inclusion-body myositis muscle fibers—Possibly caused by endoplasmic reticulum stress. Neurosci Lett. 474, 140-143.
Nogalska, A., Engel W. K., McFerrin J., Kokame K., Komano H., Askanas V., 2006. Homocysteine-induced endoplasmic reticulum protein (Herp) is up-regulated in sporadic inclusion-body myositis and in endoplasmic reticulum stress-induced cultured human muscle fibers. J. Neurochem. 96, 1491-9.
Nogalska, A., Terracciano C., D'Agostino C., Engel W. K., Askanas V., 2009. p62/SQSTM1 is overexpressed and prominently accumulated in inclusions of sporadic inclusion-body myositis muscle fibers, and can help differentiating it from polymyositis and dermatomyositis. Acta Neuropathol. 118, 407-13.
Nogalska, A., Wojcik S., Engel W. K., McFerrin J., Askanas V., 2007. Endoplasmic reticulum stress induces myostatin precursor protein and NF-kappaB in cultured human muscle fibers: relevance to inclusion body myositis. Exp Neurol. 204, 610-8.
Ono, K., Ikemoto M., Kawarabayashi T., Ikeda M., Nishinakagawa T., Hosokawa M., et al., 2009. A chemical chaperone, sodium 4-phenylbutyric acid, attenuates the pathogenic potency in human alpha-synuclein A30P+A53T transgenic mice. Parkinsonism Relat Disord. 15, 649-54.
Papp, E., Csermely, P., 2006. Chemical chaperones: mechanisms of action and potential use. Handb Exp Pharmacol. 405-16.
Ricobaraza, A., Cuadrado-Tejedor M., Garcia-Osta A., 2011. Long-term phenylbutyrate administration prevents memory deficits in Tg2576 mice by decreasing Abeta. Front Biosci. 3, 1375-84.
Ricobaraza, A., Cuadrado-Tejedor M., Perez-Mediavilla A., Frechilla D., Del Rio J., Garcia-Osta A., 2009. Phenylbutyrate ameliorates cognitive deficit and reduces tau pathology in an Alzheimer's disease mouse model. Neuropsychopharmacology. 34, 1721-32.
Roy, A., Ghosh A., Jana A., Liu X., Brahmachari S., Gendelman H. E., Pahan K., 2012. Sodium phenylbutyrate controls neuroinflammatory and antioxidant activities and protects dopaminergic neurons in mouse models of Parkinson's disease. PLoS ONE. 7, e38113.
Rubenstein, R. C., Egan M. E., Zeitlin P. L., 1997. In vitro pharmacologic restoration of CFTR-mediated chloride transport with sodium 4-phenylbutyrate in cystic fibrosis epithelial cells containing delta F508-CFTR. J Clin Invest. 100, 2457-65.
Saido, T., Leissring, M. A., 2012. Proteolytic Degradation of Amyloid beta-Protein. Cold Spring Harb Perspect Med. 2, a006379.
Selkoe, D. J., 2011. Resolving controversies on the path to Alzheimer's therapeutics. Nat Med. 17, 1060-5.
Shacka, J. J., Klocke B. J., Shibata M., Uchiyama Y., Datta G., Schmidt R. E., et al., 2006. Bafilomycin Al inhibits chloroquine-induced death of cerebellar granule neurons. Mol Pharmacol. 69, 1125-36.
Terracciano, C., Nogalska A., Engel W. K., Askanas V., 2010. In AbetaPP-overexpressing cultured human muscle fibers proteasome inhibition enhances phosphorylation of AbetaPP751 and GSK3beta activation: effects mitigated by lithium and apparently relevant to sporadic inclusion-body myositis. J. Neurochem. 112, 389-396.
Terracciano, C., Nogalska A., Engel W. K., Wojcik S., Askanas V., 2008. In inclusion-body myositis muscle fibers Parkinson-associated DJ-1 is increased and oxidized. Free Radic Biol Med. 45, 773-9.
Vattemi, G., Engel W. K., McFerrin J., Askanas V., 2004. Endoplasmic reticulum stress and unfolded protein response in inclusion body myositis muscle. Am J. Pathol. 164, 1-7.
Vattemi, G., Engel W. K., McFerrin J., Buxbaum J. D., Pastorino L. Askanas V., 2001. Presence of BACE1 and BACE2 in muscle fibres of patients with sporadic inclusion-body myositis. Lancet. 358, 1962-4.
Vattemi, G., Nogalska A., Engel W. K., D'Agostino C., Checker F., Askanas V., 2009. Amyloid-beta42 is preferentially accumulated in muscle fibers of patients with sporadic inclusion-body myositis. Acta Neuropathol. 117, 569-74.
Yang, C. C., Alvarez R. B., Engel W. K., Askanas V., 1996. Increase of nitric oxide synthases and nitrotyrosine in inclusion-body myositis. Neuroreport. 8, 153-8.
Yankner, B. A., Lu, T., 2009. Amyloid beta-protein toxicity and the pathogenesis of Alzheimer disease. J Biol Chem. 284, 4755-9.
Zhou, W., Bercury K., Cummiskey J., Luong N., Lebin J., Freed C. R., 2011. Phenylbutyrate up-regulates the DJ-1 protein and protects neurons in cell culture and in animal models of Parkinson disease. J Biol Chem. 286, 14941-51.

1. A method for treating s-inclusion body myositis, s-IBM, or ameliorating one or more pathological phenotypes of s-IBM comprising administering to a patient in need thereof an effective amount of phenylbutyrate.

2. The method of claim 1, wherein the phenylbutyrate is sodium phenylbutyrate or glycerol-phenylbutyrate.

3. The method of claim 1, wherein the phenylbutyrate is administered in an amount effective to reduce vacuolization or eliminate or slow the formation of vacuolization in the patient.

4. The method of claim 1, wherein the phenylbutyrate is administered in an amount effective to reduce Aβ42, or eliminate or slow the formation of Aβ42 in the patient.

5. The method of claim 1, wherein the phenylbutyrate is administered in an amount effective to reduce Aβ42 oligomers, or eliminate or slow the formation of Aβ42 oligomers in the patient.

6. The method of claim 1, wherein the phenylbutyrate is administered in an amount effective to decreasing the activity of γ-secretase in the patient.

7. A method of improving lysosomal activity and/or ameliorating a pathological phenotype associated with impaired autophagy in a patient in need thereof comprising:
administering to the patient an effective amount of phenylbutyrate.

8. The method of claim 7, wherein the phenylbutyrate comprises at least one of sodium phenylbutyrate or glycerol-phenylbutyrate.

9. The method of claim 8, wherein the pathological phenotype is present in muscle cells of the patient, and the amount administered is effective to ameliorate the pathological phenotype in the muscle cells.

10. The method of claim 9, wherein the administered amount is effective to reduce, eliminate or slow the formation of vacuolization in the patient treated.

11. The method of claim 9, wherein the administered amount is effective to reduce Aβ42 in the patient, or eliminate or slow the formation of Aβ42 in the patient.

12. The method of claim 9, wherein the amount administered is effective to reduce Aβ42 oligomers in the patient, or slow or eliminate the formation of the formation of Aβ42 oligomers in the patient.

13. The method of claim 8, where the amount administered is effective to improve the lysosomal activity in muscle cells of the patient.

14. The method of claim 10, wherein the improved lysosomal activity is an increase in an activity of at least one lysosomal protease selected from the group of Cathepsin D and Cathepsin B.

15. A method of improving lysosomal activity and/or ameliorating a pathological phenotype in a cell having impaired autophagy comprising:
treating the cell with an effective amount of 4-phenylbutyrate.

16. The method of claim 15, wherein the phenylbutyrate comprises at least one of sodium phenylbutyrate or glycerol-phenylbutyrate.

17. The method of claim 16, wherein the cell is a muscle cell.

18. The method of claim 17, wherein the administered amount is effective to reduce, eliminate or slow the formation of vacuolization in the cell.

19. The method of claim 17, wherein the administered amount is effective to reduce Aβ42 in the cell, or eliminate or slow the formation of Aβ42 in the cell.

20. The method of claim 17, wherein the amount administered is effective to reduce Aβ42 oligomers in the cell, or slow or eliminate the formation of the formation of Aβ42 oligomers in the cell.

21. The method of claim 17, where the amount administered is effective to improve the lysosomal activity in the cell.

22. The method of claim 17, wherein the improved lysosomal activity is an increase in an activity of at least one lysosomal protease selected from the group of Cathepsin D and Cathepsin B.

23. The method of claim 22, wherein the improved lysosomal activity is an increase in both Cathepsin D and Cathepsin B.
